(19) United States
(12) Patent Application Publication
(10) Pub. No.: US 2010/0078307 A1
(43) Pub. Date: Apr. 1, 2010

(54) Anaerobic process for treating organic material to generate biogas

(76) Inventors: Parker Dale, Newport Beach, CA (US); Parker David Dale, New York, NY (US); Jay M. Johnston, Pottsville, PA (US)

Correspondence Address: ROBERT J. BARAN, ESQ.;ATTORNEY OF RECORD, 2372 S.E. BRISTOL STREET, SUITE B, NEWPORT BEACH, CA, 92660-0755, US

(21) Appl. No.: 12/586,126

(22) Filed: Sep. 17, 2009

Related U.S. Application Data
(60) Provisional application No. 61/192,357, filed on Sep. 18, 2008.

(51) Int. Cl. C01B 3/22; C12P 1/04

(52) U.S. Cl. 204/157.52; 435/41

(57) ABSTRACT

The present invention provides an anaerobic digestion process for the treatment of organic waste materials, which process comprises a bacterial process that is carried out in the absence of oxygen and wherein said process comprises digestion, in which said waste is fermented in tanks at an elevated temperature, and wherein said process results in the production of biogas, which can be used in generators for electricity production and/or in boilers for heating purposes, the comprises treating an organic waste with a composition comprising a fermentation supernatant containing active enzymes from a Saccharomyces cerevisiae culture; and a non-ionic surfactant, wherein said nonionic surfactant may be selected from the group consisting of ethoxylated nonylphenol and ethoxylated octyl phenol.

### BACKGROUND OF THE INVENTION

1. Field of the Invention

The invention relates to a process for biologically treating a fluid which contains organic materials, in particular sewage sludge from the treatment of municipal waste waters and the like, under anaerobic conditions, to remove volatile solids and generate biogas.

2. Description of the Related Art

Since the passage of the Clean Water Act many industries have been required to institute treatment programs for the waste water they generate before these waters are discharged into public drains and waterways. These programs often include on-site waste water treatment processes, discharge into public treatment works or both.

Waste water is the term used for water which has been changed after household, commercial and industrial use, in particular water which is contaminated and flows and passes into the drainage channels.

Waste water typically contains a wide variety of contaminants which must be removed prior to discharge into public waterways and such contaminants include: organic matter, such as proteins, carbohydrates and lipids; chemicals, such as pesticides, insecticides, heavy metals and fertilizers; and sewage. The waste water is typically assessed in terms of its biochemical oxygen demand (BOD), total suspended solids (TSS) and dissolved oxygen (DO). Another important class of constituents that must be removed from waste water is the volatile organic comprises compounds (VOC) which cause or contribute to the odor of waste water.

A number of processes have been developed which are directed at specific contaminants found in waste water, for example: phenol oxidases and hydrogen peroxide have been used to decolorize pulp and paper mill waste water (U.S. Pat. No. 5,407,577); enzymes from an atypical strain of Bacillus stearothermophilus have been used to degrade algal cell walls (U.S. Pat. No. 5,139,945); a combination of bacteria and enzymes have been used to improve the water quality of standing bodies of water (U.S. Pat. No. 5,227,067); cellulases have been used to digest wood/paper compositions (U.S. Pat. No. 5,326,477); Xanthomonas maltophilia and Bacillus thuringiensis have been used to degrade polar organic solvents (U.S. Pat. No. 5,369,031); yeast has been used to digest carbohydrate-containing waste water (U.S. Pat. No. 5,075,008); a combination of beta.-glucanase, alpha.-amylase and proteases have been used to digest microbial slime (U.S. Pat. No. 5,071,765); and a combination of amylase, lipase and/or proteases have been used to digest colloidal material such as starch, grease, fat and protein (U.S. Pat. No. 5,885,950). However, each of these compositions are directed at only a specific contaminant and they do not address the variety of contaminants which are usually found in waste water and other polluted water. A composition described in U.S. Pat. No. 3,635,797 used a yeast fermentation composition to deodorize sewage ponds and degrade organic waste. However, this composition has been found to be unstable and yielded variable results from one batch to another.

The above processes are generally carried out under aerobic conditions, that is, the treating process requires the presence of oxygen, usually from air.

The present inventors have developed a liquid composition comprising a fermentation supernatant containing active enzymes from a Saccharomyces cerevisiae culture; preservatives selected from the group consisting of sodium benzoate, imidazolidinyl urea, diazolidinyl urea and mixtures thereof; calcium chloride; and a non-ionic surfactant selected from the group consisting of ethoxylated alkylphenols. This liquid composition has been used under aerobic conditions to treat, among other liquids, municipal sewage. (See U.S. Pat. Nos. 5,820,758; 5,849,566; 5,879,928 and 5,885,590.)

The biological treatment of liquids contaminated with organic materials or the purification of waste water to remove organic contaminants, which contaminants are contained in the liquids in a dissolved, colloidal or finely dispersed form, by microbial activity, e.g. by anaerobic degradation, generates a combustible gas, known as biogas.

Generally, waste water is biologically purified in waste treatment plants using the same or similar procedures which occur when the waste water biologically cleans itself in running waters, i.e. under aerobic conditions, albeit, in a technically more intensive manner. In nature, the anaerobic process of biological purification likewise occurs, e.g. at the bottom of flat, still waters.

For the purposes of defining the present invention the terms ‘anaerobic degradation’, ‘anaerobic process’, ‘anaerobic conditions’ etc. are understood to mean the conversion of organic materials by means of micro-organisms, e.g. bacteria, while excluding oxygen. As stated above, during the process of anaerobic degradation of organic materials, biogas is produced, i.e. a gas mixture which consists of methane, mainly, and carbon dioxide and traces of other ingredients.

Methods for biologically treating liquids, containing high amounts of organic materials as contaminants, under anaerobic conditions are known for treating waste waters from the foodstuff industry, agriculture, mineral oil industry as well as from pulp making. In other words, they it is possible to treat many liquids but, in general, such known biological methods are incapable of providing a full purification or complete conversion of such organic contaminants.

It is one object of this invention to treat an organic waste material, in a bacterial process, while excluding oxygen, by digesting said waste at an elevated temperature to produce biogas, which biogas can be used in generators for electricity production and/or in boilers for heating purposes.

It is another object of the invention to treat sewage sludge in a bacterial process that is carried out while excluding oxygen by fermenting said sludge at an elevated temperature to produce a biogas, which can then be used in generators for electricity production and/or in boilers for heating purposes and, in particular said biogas may be used to provide the heat to treat said sewage sludge.

It is another object of the invention to treat sewage sludge in a bacterial process that is carried out, while excluding oxygen, by fermenting said sludge at an elevated temperature to reduce the volatile organic solids (VOS).

It is another object of the invention to treat sewage sludge in a bacterial process that is carried out, while excluding oxygen, by fermenting said sludge at an elevated temperature to reduce the weight and/or volume of the treated, solid sludge product leaving the process.

Other objects of this invention will become apparent from a reading of the present specification.

### SUMMARY OF THE INVENTION

The present invention provides a process for the treatment of organic waste materials, which process comprises a bacterial process that is carried out under anaerobic conditions, i.e. in the absence of oxygen, and wherein said process comprises digestion, in which said waste is fermented in tanks at an elevated temperature, and wherein said process results in the production of biogas, which can be used in generators for electricity production and/or in boilers for heating purposes. In the process of this invention, the organic waste is treated with a composition comprising a fermentation supernatant containing active enzymes from a Saccharomyces cerevisiae culture; and a non-ionic surfactant.

In a preferred embodiment of the process of this invention the organic waste comprises sewage sludge, which is treated in a bacterial process that is carried out in the absence of oxygen and wherein said process comprises, either, thermophilic digestion, in which sludge is fermented in tanks at a temperature of about 55-60° C., or mesophilic digestion, wherein said process is carried out at a temperature of about 35-40° C. The methane in biogas can be burned to produce both heat and electricity, usually with a reciprocating engine or turbine, Fuel Cells often in a cogeneration arrangement where the electricity and waste heat generated are used to warm the digesters or to heat buildings. Excess electricity can be sold to suppliers or put into the local grid. Electricity produced by anaerobic digesters is considered to be renewable energy and may attract subsidies. Biogas does not contribute to increasing atmospheric carbon dioxide concentrations because the gas is not released directly into the atmosphere and the carbon dioxide comes from an organic source with a short carbon cycle.

In the process of the invention, a combustible biogas is produced, which comprises methane, and can be used in generators for electricity production and/or in boilers for heating purposes.

In a preferred embodiment of the process of this invention said nonionic surfactant is selected from the group consisting of ethoxylated alkylphenols, e.g. said nonionic surfactant may be selected from the group consisting of ethoxylated nonylphenol and ethoxylated octyl phenol, e.g. the nonionic surfactant may be a nonyl or octyl phenol adduct comprising from 20 to 40 moles ethylene oxide, e.g. about 30 moles ethylene oxide.

### BRIEF DESCRIPTION OF THE DRAWINGS

The advantages and features of the present invention will be better understood by the following description when considered in conjunction with the accompanying drawings in which:

FIG. 1 shows, in block diagram form, the configuration of a typical plant for treating the effluent from a plant for manufacturing food.

FIG. 2 shows the effects of treating the effluent from the food manufacturing plant of FIG. 1, by the process of this invention.

### DETAILED DESCRIPTION OF THE INVENTION

Non-ionic surfactants suitable for use in the present invention include, but are not limited to, polyether non-ionic surfactants comprising fatty alcohols, alkyl phenols, fatty acids and fatty amines which have been ethoxylated; polyhydroxyl non-ionic (polyols) typically comprising sucrose esters, sorbital esters, alkyl glucosides and polyglycerol esters which may or may not be ethoxylated. In one embodiment of the present invention a surfactant of the general formulae:

H(OCH₂CH₂)ₓ(OC₆H₄R

wherein x represents the number of moles of ethylene oxide added to the alkyl phenol and R represents a long chain alkyl group, e.g. a C₇-C₁₀ normal-alkyl group and, in particular, the nonionic surfactant is an ethoxylated octyl phenol which is sold under the tradename IGEPAL CA-630, is used. The non-ionic surfactant acts synergistically to enhance the action of the yeast fermentation supernatant.

The composition of the present invention is similar to that described in U.S. Pat. No. 3,635,797 to Battistoni et al., which is hereby incorporated by reference in its entirety. Briefly, yeast, Saccharomyces cerevisiae, is cultured in a medium comprising: a sugar source, such as sucrose from molasses, raw sugar, soybeans or mixtures thereof. A sugar concentration of about 10 to about 30%, by weight; malt such as diastatic malt at a concentration of about 7 to about 12%, by weight; a salt, such as a magnesium salt, and, in particular, magnesium sulfate, at a concentration of about 1 to about 3%, by weight, and yeast are added to the medium to obtain a final concentration of about 1 to about 5%, by weight, of yeast in the final culture mixture. The mixture is incubated at about from 26 degrees to about 42 degrees C. until the fermentation is completed, i.e. until effervescence of the mixture has ceased, usually about 2 to about 5 days depending on the fermentation temperature. At the end of the fermentation the yeast fermentation composition is centrifuged to remove the “sludge” formed during the fermentation. The supernatant (about 98.59%, by weight) is mixed with sodium benzoate (about 1%, by weight), imidazolidinyl urea (about 0.01%, by weight), diazolidinyl urea (about 0.15%, by weight), calcium chloride (about 0.25%, by weight) to form the fermentation intermediate. The pH is adjusted to from about 3.7 to about 4.2 with phosphoric acid. The composition of the fermentation intermediate is summarized in Table I.

TABLE I
Fermentation Intermediate

| Component | %, by weight |
|---|---|
| Fermentation supernatant | 98.59 |
| Na Benzoate | 1 |
| Imidazolidinyl urea | 0.01 |
| Diazolidinyl urea | 0.15 |
| Calcium chloride | 0.25 |
| Adjust pH | to about 3.7 to about 4.2 with |
| | phosphoric acid |

The fermentation intermediate is prepared by filling a jacketed mixing kettle with the desired quantity of the fermentation supernatant. With moderate agitation the pH is adjusted to from about 3.7 to about 4.2 with phosphoric acid. With continuous agitation, sodium benzoate, imidazolidinyl urea, diazolidinyl urea and calcium chloride are added. The temperature of the mixture is then slowly raised to about 40 degrees C. and the mixture is agitated continuously. The temperature is maintained at about 40 degrees C. for about one hour to ensure that all the components of the mixture are dissolved. The mixture is then cooled to form about 20 degrees to about 25 degrees C.

The fermentation intermediate is then formulated into the composition of the present invention (final composition) by mixing the fermentation intermediate (about 20.24%, by weight, of the final composition) with, preservatives such as sodium benzoate, imidazolidinyl urea, diazolidinyl urea, imidazolidinyl urea, diazolidinyl urea and mixtures thereof (about 0.16%, by weight, of the final composition), a non-ionic surfactant such as ethoxylated octyl phenol (about 9%, by weight, of the final composition) and the composition is brought to 100% by the addition of water. In a preferred embodiment of the present invention the composition comprises about 20.24%, by weight, fermentation intermediate, about 0.1%, by weight, sodium benzoate, about 0.01%, by weight, imidazolidinyl urea, about 0.15%, by weight, diazolidinyl urea, about 9%, by weight, ethoxylated octyl phenol. (See Table II).

TABLE II
Final Composition

| Component | % by weight |
|---|---|
| Sodium benzoate | 0.1 |
| Imidazolidinyl urea | 0.01 |
| Diazolidinyl urea | 0.15 |
| Ethoxylated octyl phenol | 9.00 |
| Fermentation Intermediate | 20.24 |

The method for preparing the final composition is as follows: A mixing kettle is charged with the desired volume of water at about 20 degrees to about 25 degrees C. Sodium benzoate, imidazolidinyl urea and diazolidinyl urea are added while the solution is agitated. The mixture is agitated until the solids are dispersed. Ethoxylated octyl phenol is then added and the agitation is continued. The fermentation intermediate is then added with gentle agitation. The pH is adjusted to about 3.5 to about 4.0 with phosphoric acid.

After mixing and pH adjustment, the final concentration of components in the final composition are summarized in Table III.

TABLE III
Final Composition

| Component | %, by weight |
|---|---|
| Na benzoate | 0.3 |
| Imidazolidinyl urea | 0.01 |
| Diazolidinyl urea | 0.15 |
| Ethoxylated octyl phenol | 9 |
| Calcium chloride | 0.05 |
| Fermentation supernatant 20 (clarified) | |
| Adjust pH | to about 3.5 to 4.0 with |
| | phosphoric acid |

The final composition is diluted for use in a zone for anaerobic digestion. For use in treating waste water the final composition is diluted to as high as parts per million. For other uses it may desirable to dilute the final composition only as little as 1 in 10. Those skilled in the art are aware that dilutions of such compositions can be used and that over-dilution for a particular purpose can result in a decreased rate of digestion and that under-dilution for a particular purpose increases cost without increasing the rate of degradation. Ideally, the final composition is diluted to optimize the rate of degradation of a particular waste and to minimize costs.

In use, the composition of the present invention enhances the degradation of pollutants, presumably, by enhancing the activity of bacteria commonly found in waste water treatment plants and, unexpectedly, increases the amount of biogas generated, while decreasing the volatile odorous compounds (VOC) and the volume and weight of the effluent from the anaerobic zone. In an aerobic process, wherein the above final composition is utilized to degrade pollutants in the presence of bacteria, it is expected that DO is decreased as the bacteria metabolize the available oxygen and the surfactant and yeast fermentation supernatant act synergistically to enhance the rate of degradation and increase DO. In such aerobic process, the surfactant, alone, or the yeast fermentation supernatant, alone, does not result in the enhanced activity observed when they are combined.

However, in an anaerobic process it could not predictable what advantages, if any, would be obtained, by treating the organic waste material with the above-described final composition. However, like the aerobic process, the enhanced degradation observed in use of the final composition, in an anaerobic process is proportional to the time that the final composition is in contact with the waste water to be treated. Therefore, it is desirable that the final composition is added to the waste water at the earliest opportunity. Preferably, the final composition is added upstream of the anaerobic zone of the waste water treatment plant. The final composition may be added to the waste water by continuously pumping the final composition into the waste water or it may be added in batches as desired to reach the desired dilution of the final composition in the anaerobic zone.

The invention is further illustrated by the following examples which are illustrative of a specific mode of practicing the invention and are not intended as limiting the scope of the claims.

### Example 1

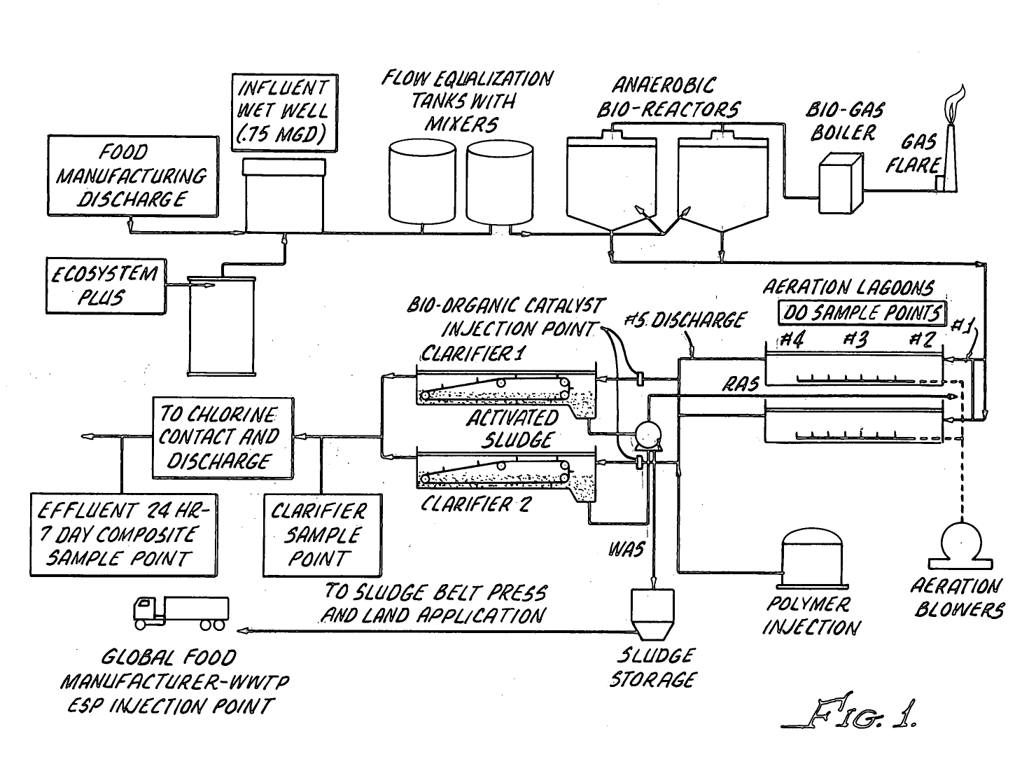

The process of the present invention may be exemplified by the treatment of the discharge from a food manufacturing plant. As shown in FIG. 1, two sequential anaerobic bioreactors are in line subsequent to the influent wet well(s) where the discharge from the food manufacturing is collected.

The flow rate is 0.75 million gallons per day (MGD). In the anaerobic bioreactors, the flow from the wet wells is contacted with the final composition described above. The ratio of the flow of waste water and the final composition varies from 0.0000667% TO 0.0002667%. After treatment in the anaerobic zone, the liquid effluent from the bioreactors is led to one or more aeration lagoons for further treatment. The gaseous effluent from the bioreactors is collected and either flared or recycled (and may be treated e.g. to increase its BTU value, prior to recycling) for use in providing heat to the bioreactors and or Food processing Boiler used to generate heat steam for the manufacturing process.

It was found that treatment of the influent to the bioreactor increased the biogas, i.e. Biomethane, from 1.53 cubic foot to 1.93 cubic foot per lb. of total chemical oxygen demand. This is a surprising increase of 26% and concomitantly the sludge volume of the effluent was reduced by 28%.

### Example 2

In a separate example of the process of this invention, the waste water from a large cheese manufacturing plant was treated in an anaerobic digestion zone with the final product of Table 3, above, at a ratio of from 0.0220 to 0.1484 final composition of Table 3 influent. The Average residence time in the anaerobic zone was 2.72 to 4.28 Day depended on Influent Flows. The temperature during said treatment was from about 94 to about 102 degrees F. In this trial, the removal rate of the TCOD increased from 29% to 73.9%. Biomethane production increased from 1000 cubic foot per hour to 1,800 cubic foot per hour. This is a surprising increase of 80%.

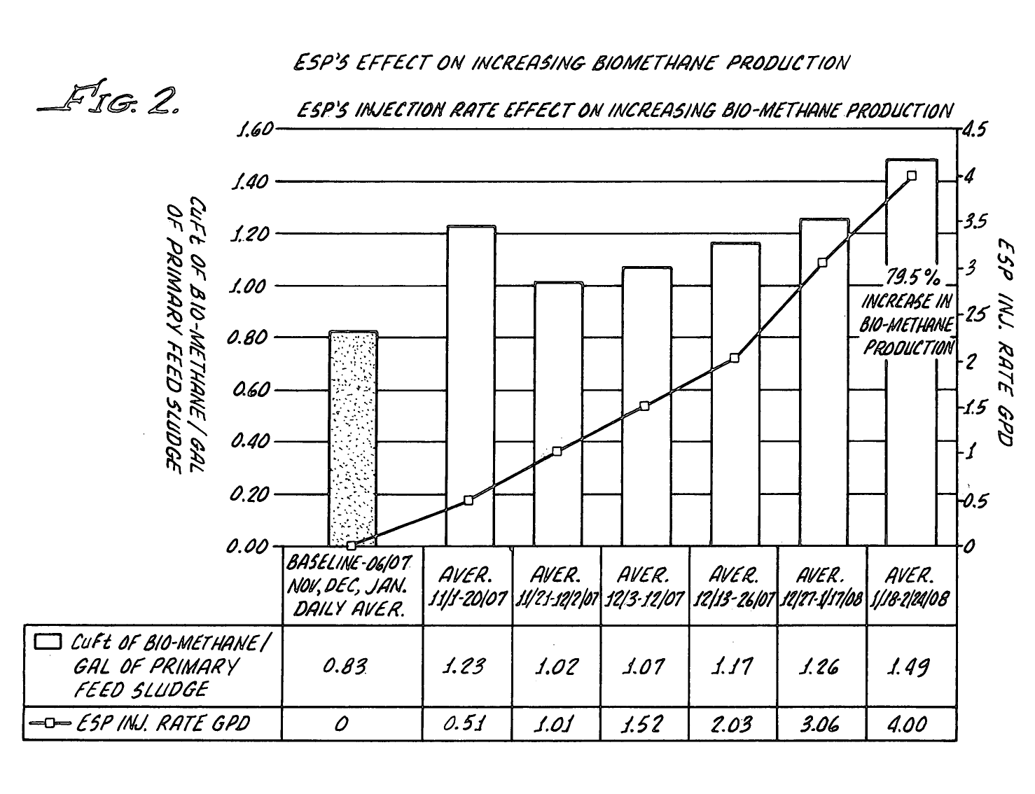

The result is reported in FIG. 2.

### Example 3

The process of the present invention was also utilized in the treatment of sewage sludge from a municipal source. In this trial the influent to the anaerobic zone of a municipal sewage treating plant was contacted with the final composition of Table 3, above, at a ratio of 0.0271 to 0.122 ESP Gals/1,000 gal Primary Feed Sludge and a temperature of 92 To 102° F. This residence time of the mixture of sewage sludge and the final composition in the anaerobic zone was 15 to 18 Days depended on Influent primary feed loading to A.D.

A typical Municipal Waste Water Treatment Facility processes 1000 gallons per day of wastewater for every person served.

Approximately 1.0 cubic foot (ft³) of digester gas is produced by an anaerobic digester per person per day.

The heating value of the biogas produced by anaerobic digesters is approximately 600 British thermal units per cubic foot (Btu/ft³).

In the present example, the following results were obtained:

T.S. Removal Rates increased by 80.9%, from 6.81% to 35.6%

T.V.S. removal rates increased by 19.2%, from 49.61% (Start of treatment with the composition of Table 3) to 61.4%

Sludge Volumes were reduced by 25%

Actual production of biogas increased 74.6%, from 0.81 cubic foot (ft³) to 1.42 per 100 gallons Influent Flow

There was an 88% increase, from 0.83 cubic foot (ft³) per gallon of primary digester feed sludge to 1.56

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. For example, while not specifically described herein, the biogas generated from the process of this invention may be used in fuel cell applications.

The Northeast Regional Biomass Program, in conjunction with XENERGY, Inc., has completed a comprehensive study examining the feasibility of utilizing bio-based fuels with stationary fuel cell technologies. The findings show that biomass-based fuel cell systems, from a technical perspective, are capable of providing a source of clean, renewable electricity over the long-term. In addition, fuel cells have proven to be successful in this application, in service around the world at several landfills and wastewater treatment plants (as well as breweries and farms), generating power from the methane gas they produce, and reducing harmful emissions in the process.

Fuel cells have been operated at landfills and wastewater treatment facilities all over the United States and in Asia. For example, Connecticut's Groton Landfill has been producing 600,000 kWh of electricity a year, with a continuous net fuel cell output of 140 kW and UTC Power's (formerly IFC/ONSI) fuel cell system at the Yonkers wastewater treatment plant in New York, produces over 1.6 million kWh of electricity per year, while releasing only 72 pounds of emissions into the environment. In Portland, Oreg., a fuel cell produces power using anaerobic digester gas from a wastewater facility, which generates 1.5 million kWh of electricity per year, substantially reducing the treatment plant's electricity bills.

Fuel Cell Energy, Inc. (FCE) is installing its Direct FuelCell® (DFC) power plants at wastewater treatment plans around the world.

Both FCE and UTC have installed fuel cells at several breweries—Sierra Nevada, Kirin, Asahi and Sapporo—using the methane-like digester gas produced from the effluent from the brewing process to power the fuel cell.

The process of the present invention can be used to generate a biogas that may be used in any of the above commercial processes to generate power from waste.

It is intended that all such modifications will fall within the scope of the appended claims.

What is claimed is:

1. In an anaerobic digestion process for the treatment of sewage sludge, which process comprises a bacterial process that is carried out in the absence of oxygen and wherein said process comprises either thermophilic digestion, in which sludge is fermented in tanks at a temperature of 55° C., or mesophilic, at a temperature of around 36° C., and wherein said process results in the production of biogas, which can be used in generators for electricity production and/or in boilers for heating purposes, the improvement comprising treating a sewage sludge resulting from the treatment of municipal or industrial waste water with a composition comprising of a fermentation supernatant comprising active enzymes from a Saccharomyces cerevisiae culture and a non-ionic surfactant.

2. The process of claim 1 wherein said composition comprises about 20%, by weight, of a fermentation supernatant comprising active enzymes from a Saccharomyces cerevisiae culture.

3. The process of claim 1 wherein said composition comprises; about 0.3%, by weight, sodium benzoate; about 0.01%, by weight, imidazolidinyl urea; about 0.15%, by weight, diazolidinyl urea.

4. The process of claim 1 wherein said composition comprises about 9%, by weight, of a non-ionic surfactant.

4. The process of claim 1 further comprising the improvement of reducing the concentration of volatile solids.

5. The process of claim 1 wherein said nonionic surfactant is an ethoxylated alkylphenol.

6. The process of claim 1 wherein said nonionic surfactant is an ethoxylated nonyl or octyl phenol.

7. The process of claim 1 wherein said treatment occurs in the absence of urea or saponins.

8. In an anaerobic digestion process for the treatment of sewage sludge, which process comprises a bacterial process that is carried out in the absence of oxygen and wherein said process comprises either thermophilic digestion, in which sludge is fermented in tanks at a temperature of 55° C., or mesophilic, at a temperature of around 36° C., and wherein said process results in the production of biogas, which can be used in generators for electricity production and/or in boilers for heating purposes, the improvement comprising treating a sewage sludge resulting from the treatment of municipal or industrial waste water with a composition consisting essentially of a fermentation supernatant containing active enzymes from a Saccharomyces cerevisiae culture; preservatives selected from the group consisting of sodium benzoate, imidazolidinyl urea, diazolidinyl urea and mixtures thereof; calcium chloride; and a non-ionic surfactant selected from the group consisting of ethoxylated alkylphenols.

9. The process of claim 8 wherein said non-ionic surfactant is selected from the group consisting of ethoxylated nonylphenol and ethoxylated octyl phenol.

10. The process of claim 9 wherein said preservatives are present at a concentration of about 0.46%, by weight; the surfactant is present at a concentration of about 9%, by weight; and the fermentation supernatant is present at a concentration of about 20%, by weight.

11. In an anaerobic digestion process for the treatment of organic waste materials, which process comprises a bacterial process that is carried out in the absence of oxygen and wherein said process comprises digestion, in which said waste is fermented in tanks at an elevated temperature, and wherein said process results in the production of biogas, which can be used in generators for electricity production and/or in boilers for heating purposes, the improvement comprising treating an organic waste with a composition comprising a fermentation supernatant containing active enzymes from a Saccharomyces cerevisiae culture; and a non-ionic surfactant.

12. The process of claim 11 wherein said nonionic surfactant is selected from the group consisting of ethoxylated alkylphenols.

13. The process of claim 12 wherein said nonionic surfactant is selected from the group consisting of ethoxylated nonylphenol and ethoxylated octyl phenol.

14. In a process for generating electricity from organic waste materials, which comprises converting a biogas generated from said organic waste to hydrogen in a fuel cell and converting said hydrogen to electricity, the improvement which comprises providing said biogas of claim 11 to said fuel cell and converting said biogas to hydrogen.
